(19) United States
(12) Patent Application Publication
(10) Pub. No.: US 2022/0218789 A1
(43) Pub. Date: Jul. 14, 2022

(54) Nogapendekin Alfa-Inbakicept For Immune Stimulant Therapies And Treatment Of Viral Infections

(71) Applicants: Nant Holdings IP, LLC, Culver City, CA (US); NantBio, Inc., Culver City, CA (US); Altor Bioscience Corporation, Miramar, FL (US)

(72) Inventors: John LEE, Topanga, CA (US); Patrick SOON-SHIONG, Los Angeles, CA (US); Shahrooz RABIZADEH, Culver City, CA (US)

(21) Appl. No.: 17/594,940

(22) PCT Filed: May 8, 2020
(86) PCT No.: PCT/US2020/032211

§ 371 Date: Nov. 3, 2021

Related U.S. Application Data
(60) Provisional application No. 62/985,074, filed on Mar. 4, 2020.
(60) Provisional application No. 62/984,085, filed on Mar. 2, 2020.
(60) Provisional application No. 62/904,019, filed on Sep. 23, 2019.
(60) Provisional application No. 62/846,186, filed on May 10, 2019.

(51) Int. Cl. A61K 38/17; A61P 31/16; A61K 45/06

(52) U.S. Cl. CPC A61K 38/1793; A61K 45/06; A61P 31/16

(57) ABSTRACT

Pharmaceutical compositions comprising an IL-15 agonist or derivative thereof, such as nogapendekin alfa-inbakicept (NAI), are provided herein for enhancing immunity and for inhibiting viral infections. The IL-15 agonist or derivative thereof may be used to increase proliferative capacity and/or cytotoxicity of various immune competent cells, and especially NK and cytotoxic T cells in healthy individuals. The IL-15 agonist or derivative thereof may be used alone or in combination with one or more other therapeutic agents, such as in conjunction with a vaccine.

### FIELD

The present invention generally relates to IL-15 agonist compounds, pharmaceutical compositions thereof, and uses thereof in immune stimulant therapies, such as preventing or treating a viral infection or cancer, and enhancing immunity, particularly in healthy individuals.

### BACKGROUND

The background description includes information that may be useful in understanding the present disclosure. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

IL-15 is a pro-inflammatory cytokine that plays a critical role in the innate and adaptive immune responses. Many immune cells, including lymphocytes (T and B cells), NK cells, monocytes, macrophages, dendritic cells, neutrophils, eosinophils, and mast cells express the IL-15 receptor, IL-15Rα, and are sensitive to IL-15 in the microenvironment. Binding of IL-15 to IL-15Rα activates the intracellular JAK/STAT signaling pathway, leading to systemic immunomodulatory effects. IL-15 plays a role in the innate and adaptive immune response to various viral infections, including HIV, HTLV, herpesvirus, and hepatitis infections. In vivo, IL-15 is typically trans-presented with IL-15Rα to activate immune cells.

U.S. 2009/0238791 reports an IL-15/IL-15Rα fusion protein comprising an IL-15Rα sushi domain.

U.S. 2013/0142755 reports an IL-15/IL-15Rα-IgG1-Fc complex that activates NK, NTK and memory CD8⁺ T cells.

U.S. Pat. No. 9,328,159 reports an IL-15/IL-15Rα-IgG1-Fc complex where the IL-15 contains an N72D point mutation. This superagonist is also known as nogapendekin alfa-inbakicept (NAI), and previously known as ALT-803 or N-803.

WO 2017/210649 reports that NAI can be an adjuvant for HPV vaccines.

Additionally, the safety, tolerability, pharmacokinetics, and immunologic effects of intravenous and subcutaneous NAI were evaluated in a human Phase I trial of patients with advanced solid tumors (Clin Cancer Res Apr. 1 2019; DOI:10.1158/1078-0432). Unfortunately, this trial did not observe single agent benefit in their study population. In other known uses, IL-15N72D:IL-15RαSu/IgG1 Fc complexes and modified forms thereof were reported to enhance an immune response against a neoplasia or a viral infection as is described in US 2019/0023766. In still further known uses, IL-15N72D:IL-15RαSu/IgG1 Fc complexes were used in conjunction with various therapeutic antibodies (e.g., rituximab) as is described in WO 2016/004060. Notably, however, use of the IL-15N72D:IL-15RαSu/IgG1 Fc complexes to prevent disease and/or enhance longevity in subjects not diagnosed with a disease have not been reported.

Thus, even though various immune stimulants are known in the art, effective use of such stimulants in the prevention of disease and/or enhancement of longevity has not been shown. Consequently, there is a need to provide improved compositions and methods for immune stimulants to treat or prevent disease and/or enhance longevity.

### SUMMARY

Various compositions and methods that employ IL-15-based therapeutics for enhancing an immune response or preventing or reducing the likelihood of cancer are described herein comprising administering to a healthy individual, a composition comprising an IL-15 agonist or derivative thereof at a dose that increases the proliferative capacity and/or cytotoxicity of a NK cell or a T cell.

In some embodiments, a method of treating age-related decline of activity of immune competent cells in a healthy individual is described herein that includes a step of administering to the individual a composition that includes an IL-15 agonist or derivative thereof at a dose that increases proliferative capacity and/or cytotoxicity of a NK cell or a T cell.

Typically, but not necessarily, the healthy individual is at least 50, or at least 55, or at least 60 years of age. Moreover, it is contemplated that the healthy individual may have a total T cell count and/or total NK cell count that is at or below a bottom quartile for a reference range for the total T cell count. Alternatively or additionally, the healthy individual may have a total memory cell count that is at or below a bottom quartile for a reference range for the total memory cell count.

In additional embodiments, a method of preventing or reducing likelihood or occurrence of an infectious disease or cancer in a healthy individual that includes a step of administering to the individual a composition that includes an IL-15 agonist or derivative thereof at a dose that increases proliferative capacity and/or cytotoxicity of a NK cell or a T cell.

Preferably, the individual is a healthcare professional, a teacher, or at least 50 years of age, and/or the individual is predisposed to infectious diseases or cancer and/or has a family history of predisposition to infectious diseases or cancer. Moreover, it is contemplated that the IL-15 agonist or derivative thereof may be administered to increase a total T cell and/or total NK cell count by at least 10%.

In additional embodiments, a method of enhancing longevity in an individual is described herein comprising administering to the individual a composition that includes an IL-15 agonist or derivative thereof at a dose that increases proliferative capacity and/or cytotoxicity of a NK cell or a T cell. Advantageously, contemplated compositions can also be administered to an individual to help induce immune memory formation prior to a therapeutic intervention that is known to compromise immune memory formation.

In additional embodiments, a method of enhancing vaccine protection of a healthy individual is described comprising administering to the individual a vaccine composition that comprises an antigen of a pathogenic virus or pathogenic bacterium, and a further step of administering an IL-15 agonist or derivative thereof to the healthy individual within equal or less than 14 days from the step of administering the vaccine composition.

For example, contemplated pathogenic viruses include an influenza virus, a morbillivirus, a rubeola virus, a rubella virus, a varicella virus, a hemorrhagic fever virus, an enterovirus, and a hepatitis virus, and contemplated pathogenic bacterium include Mycobacterium tuberculosis, Corynebacterium diphtheriae, Clostridium tetani, Bordetella pertussis, Haemophilus influenzae type B, Vibrio cholerae, Salmonella Typhi, or Streptococcus pneumoniae.

In additional embodiments, a method of suppressing viral activation in an individual infected with a latent virus is described herein. Most typically, such method will include a step of administering to the individual a composition that includes an IL-15 agonist or derivative thereof at a dose that prevents viral activation in the individual.

Among other things, the latent virus can be a dormant retrovirus that is integrated into a host cell genome of the individual, or a dormant varicella zoster virus that is integrated into a host cell of the individual, or a human papillomavirus that is integrated into a host cell of the individual.

In additional embodiments, a method of treating an individual prior to administration of an immune compromising therapy is described that includes a step of administering to the individual a composition that includes an IL-15 agonist or derivative thereof at a dose that stimulates formation of memory NK cells, T_{CM} central memory cells, T_{SCM} stem cell memory cells, and/or T_{EM} effector memory cells.

For example, in such methods it is contemplated that the immune compromising therapy is radiation therapy or chemotherapy. Where desirable, such methods may also include a step of subjecting the individual to a leukapheresis to collect lymphocytes for transfusion to the individual after the immune compromising therapy.

In additional embodiments, methods of preventing, reducing the occurrence of, or treating a viral infection in a subject in need thereof are described herein comprising administering an IL-15 agonist or derivative thereof, or a composition comprising the IL-15 agonist or derivative thereof, to the subject. In a particular embodiment, the viral infection is not an HIV or an HPC infection.

In some embodiments, the viral infection may be caused by a DNA virus, such as varicella zoster virus, or an RNA virus, such as an influenza virus. In further embodiments, the viral infection is not caused by HIV virus and/or HPV virus.

In some embodiments, the viral infection may be cause by a RNA virus, such as coronavirus. In a particular embodiment the viral infection may be caused by coronavirus disease 2019 (“COVID-19”; previously known as 2019-nCoV), and/or severe acute respiratory syndrome coronavirus 2 (“SARS-CoV-2”).

In a particular embodiment, the IL-15 agonist or derivative thereof is NAI.

The IL-15 agonists, or derivatives thereof, may be used alone or in combination with additional active pharmaceutical ingredients. They may be formulated into pharmaceutical compositions and used in in vitro and in vivo methods to treat or otherwise inhibit disease or enhance an immune response. Additionally, the IL-15 agonists, or derivatives thereof, may be used prophylactically alone or in combination with vaccine or other therapeutics.

Various objects, features, aspects, and advantages will become more apparent from the following detailed description of preferred embodiments, along with the accompanying drawing in which like numerals represent like components.

### BRIEF DESCRIPTION OF THE DRAWINGS

FIG. 1 is a representation of NAI (N-803).

### DETAILED DESCRIPTION

### I. Definitions

The following definitions refer to the various terms used above and throughout the disclosure.

The term “IL-15 agonist or derivative thereof” refers to a compound or complex that binds to and activates the IL-15 receptor (“IL-15Rα”). The type of compound or complex of the IL-15 agonist is not particularly limited so long as it binds to and activates the IL-15Rα. It is generally preferred that an IL-15 agonist or derivative thereof will include at least portions of human sequences for IL-15 and/or IL-15 Ra. Further, the IL-15 agonist or derivative thereof may have a serum half-life that is longer than isolated/recombinant and purified IL-15 alone. The IL-15 agonist may be a peptide, protein, small molecule (e.g., a pharmaceutical drug), or oligonucleotide. The peptide or proteins may be a single amino acid sequence or two or more sequences bound via covalent attachments (e.g., disulfide bonds) or non-covalent attachments (e.g., hydrophilic or hydrophobic interactions, hydrogen bonds). In a particular embodiment, the IL-15 agonist is an antibody, modified antibody, chimeric antibody, or a derivative thereof. In a further embodiment, the IL-15 agonist is a superagonist complex, such as an IL-15 derivative bound to an IL-15Rα/IgG1 Fc fusion protein, also known as NAI. NAI is also known in the literature as N-803 or ALT-803. U.S. Pat. No. 9,328,159 describes NAI and is incorporated herein by reference in its entirety. Other examples contemplated herein include P22339 (a complex of IL-15 and the Sushi domain of IL-15Rα chain with a disulfide bond linking the IL-15/Sushi domain complex with an IgG1 Fc to augment its half-life; see Nature, Scientific Reports (2018) 8:7675), and XmAb24306, which is a IL-15/IL-15Rα-Fc heterodimer (see e.g., WO 2018/071919).

Binding of the IL-15 agonist to the IL-15Rα induces a signal to downstream elements to activate the IL-15 signaling pathway and activate the cell. Cells expressing IL-15Rα include, but are not limited to, T cells, NK cells, monocytes, macrophages, dendritic cells, keratinocytes, fibroblasts, myocytes, and nerve cells. Guo, et al. Cytokine Growth Factor Rev., 2017. Binding of the IL-15 agonist to the IL-15Rα propagates a signal through the IL-15Rα (e.g., via a conformational change) that initiates the IL-15 signaling pathway to activate an immune response, such as an antiviral response.

The term “derivative” refers to a compound that is structurally similar to the reference compound such that it retains some, all, or more of its biological effect. For example, a derivative of an IL-15 agonist includes compounds or molecules that partially activate the IL-15Rα (e.g., a partial agonist), fully activate the IL-15Rα (e.g., a full agonist), or activate the IL-15Rα to a higher degree than the reference compound or molecule (e.g., a super agonist).

The term “virus” is not particularly limited and refers to both DNA and RNA viruses. The DNA virus may be single- or double-stranded viruses and may belong to any family of DNA viruses, including, but not limited to, herpesviridae, adenoviridae, polyomavididae, and poxviridae. Particular embodiments of DNA viruses include the human herpesvirus and varicella zoster virus. In some embodiments, the DNA virus is not human papillomavirus (HPV). The RNA virus may also be single- or double-stranded and may belong to any family of RNA viruses, including, but not limited to, reoviridae, coronaviridae, picornaviridae, flaviviridae, hepeviridae, togaviridae, filoviridae, paramyxoviridae, pneumoviridae, rhabdoviridae, hantaviridae, and orthomyxoviridae. Particular embodiments of RNA viruses include rotavirus, coronavirus (COVID-19; SARS-CoV-2), SARS virus, poliovirus, rhinovirus, hepatitis A virus, yellow fever virus, west nile virus, hepatitis C virus, dengue fever virus, zika virus, rubella virus, sindbis virus, Chikungunya virus, Ebola virus, Marburg virus, measles virus, mumps virus, respiratory syncytial virus, rabies virus, influenza virus A, influenza virus B, influenza virus C, and influenza virus D. In some embodiments, the virus is not human immunodeficiency virus (HIV).

The term “active pharmaceutical ingredient” or API refers to compounds having an antiviral property or are otherwise therapeutic but are not IL-15 agonists. Such APIs include, but are not limited to, agents that interfere with viral processes for binding to a cell, entering a cell, reverse transcription, viral genome replication, viral genome integration into host genome, viral transcription, viral translation, viral particle assembly, and/or viral release from the cell. APIs can be formulated in the same formulation as the IL-15 agonist, or in a different formulation for concurrent or sequential administration with the IL-15 agonist.

The term “treat” and “treatment” refers to a method for reducing, inhibiting, or otherwise ameliorating a viral infection by administering a therapeutically effective amount of an IL-15 agonist. Treating a viral infection includes interfering with a virus's capacity to bind to a cell, enter a cell, reverse transcribe its genome (if the virus is an RNA virus), replicate its genes or genome, integrated its genes or genome in the host genome, transcribe its genes, translate its genes, assemble its particles, and/or release virions from an infected cell. This can be accomplished via direct effect on the virus itself, an effect on the infected cell, or an effect on immune cells to detect and remove viral particles, virions, and/or cells infected with the virus.

The term “administering” refers to both direct and indirect administration of a pharmaceutical composition or drug, wherein direct administration of the pharmaceutical composition or drug is typically performed by a health care professional (e.g., physician, nurse, etc.), and wherein indirect administration includes a step of providing or making available the pharmaceutical composition or drug to the health care professional for direct administration (e.g., via injection, infusion, oral delivery, topical delivery, etc.).

The term “concomitant” or “concomitantly” includes administering an agent (e.g., IL-15 agonist) in the presence of a further agent. Concomitant administration in a therapeutic treatment method includes methods in which a first, second, third, or additional agents are co-administered. Concomitant administration also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may administer to the subject a second agent (e.g., IL-15 agonist), and the administering steps may be executed at the same time, or nearly the same time. The actor and the subject may be the same entity (e.g., human). Thus, the term embraces both simultaneous administration and substantially simultaneous administration, i.e., at about the same time.

The term “sequential administration” means not at the same time and means not almost at the same time. For example, one drug (active agent) may be taken at one time of day (e.g. in the morning) and the other taken at another time of day (e.g. in the evening/night time); or alternating days, etc. . . . .

### II. Uses

It has now been discovered that an IL-15 agonist or derivative thereof, and especially N-803, can be used in healthy individuals to achieve a variety of desirable outcomes, and particularly to reduce or prevent infection and/or cancer, and to so enhance longevity. Most preferably, it is contemplated that N-803 is administered subcutaneously at repeated and relatively moderate dosage levels over extended periods of time. For example, N-803 may be administered quarterly or twice a year to an ageing individual to restore decreasing levels of proliferative and cytotoxic activity of NK and T cells. In that context, it should be noted that the term “healthy individual” as used herein refers to an individual that is not seeking treatment for a previously not treated disease at the time of prescription or administration of contemplated compositions. Thus, the term “healthy individual” also includes individuals that may have been previously diagnosed with a disease (e.g., dyslipidemia, type II diabetes, etc.) for which treatment was then advised, prescribed, or administered.

Among other advantages, it should be appreciated that administration of an IL-15 agonist or derivative thereof, and especially N-803, to healthy individuals may at least partially revert an age-related decline of activity of immune competent cells. For example, where the individual is at least 50 years of age, or at least 55 years of age, or at least 60 years of age, proliferative capacity and/or cytotoxicity of NK cells and CD4⁺/CD8⁺ T cells is frequently diminished. Such reduction in immune competent cells may be detected by various measures well known in the art (see e.g., Data in Brief 12 (2017) 400-404). Among other measures, the healthy individual may have a total T and/or NK cell count that is at or below the bottom tertile, quartile, or quintile for a reference range for the total T cell and/or NK cell count (or even below the reference range). Likewise, counts for subsets of these cells may also be at or below the bottom tertile, quartile, or quintile for a reference range for the total T cell and/or NK cell count (or even below the reference range), and contemplated subsets include memory NK cells (CD16⁺, CD56⁺, or CD16^{low}, CD56^{high}), T_{CM} central memory cells, T_{SCM} stem cell memory cells, T_{EM} effector memory cells, and various B cells (CD19⁺, CD27⁺, etc.). Likewise, proliferation assays and/or cytotoxicity assays for each of the immune competent cells may be performed using protocols well known in the art.

Therefore, it should also be appreciated that administration of an IL-15 agonist or derivative thereof, and especially N-803, to healthy individuals may prevent or reduce the likelihood of an infectious disease or cancer in a healthy individual as not only cells for an innate immune response but also cells for an adaptive immune response and memory cells are significantly increased in number and/or activity. Individuals that may particularly benefit from such methods include healthcare professionals (e.g., physicians, nurses, other hospital personnel), teachers, professional individuals facing the public, and ageing individuals (e.g., those at least 50 years of age). Likewise, it should be recognized that contemplated methods will also benefit those that are predisposed to infectious diseases or cancer and/or that have a family history of predisposition to infectious diseases or cancer.

Consequently, the uses presented herein will also beneficially enhance longevity in an individual. As used herein, the term “enhance longevity” refers to an increase in disease-free time per time unit (typically at least per three or per five year interval at an age of at least 50 or at least 60 years) as compared to an age-adjusted reference population without administration of an IL-15 agonist or derivative thereof. Most typically, the disease free time will be with regard to viral disease, bacterial disease, and/or cancer.

Viewed form yet another perspective, it should be noted that administration of an IL-15 agonist or derivative thereof can also confer enhancement of vaccine protection of a healthy individual. More particularly, such methods will include administration of a vaccine composition to the individual, where the vaccine composition includes an antigen of a pathogenic virus or a pathogenic bacterium. Most typically, an IL-15 agonist or derivative thereof is administered to the healthy individual within equal or less than 14 days from the administration of the vaccine composition. For example, an IL-15 agonist or derivative thereof can be administered to the individual within that time frame (e.g., equal or less than 14 days, or equal or less than 10 days, or equal or less than 7 days, equal or less than 5 days, or equal or less than 3 days) before administration of the vaccine composition and/or after administration of the vaccine composition. Consequently, it should be noted that vaccination efficiency can be boosted by increasing proliferation capacity of various immune competent cells as well as by stimulation of immune memory formation.

For example, suitable vaccine compositions may include one or more antigens from an influenza virus, a morbillivirus, a rubeola virus, a rubella virus, a varicella virus, a hemorrhagic fever virus, an enterovirus, and a hepatitis virus, or one or more antigens from Mycobacterium tuberculosis, Corynebacterium diphtheriae, Clostridium tetani, Bordetella pertussis, Haemophilus influenzae type B, Vibrio cholerae, Salmonella Typhi, or Streptococcus pneumoniae.

In additional embodiments, an IL-15 agonist or derivative thereof may be used to suppress viral activation in an individual infected with a latent virus. Thus, the individual may be asymptomatic at the time of administration of an IL-15 agonist or derivative thereof. For example, where the latent virus is a dormant retrovirus that is integrated into a host cell genome of the individual (e.g., HIV virus), administration of an IL-15 agonist or derivative thereof will help eliminate latent infected cells, likely due to NK cell activity, before viral replication and budding will take place. On the other hand, where the latent virus is a dormant varicella zoster virus that is integrated into a host cell of the individual, a shingles outbreak may be prevented or delayed by administration of an IL-15 agonist or derivative thereof. Similarly, where the latent virus is a human papillomavirus that is integrated into a host cell of the individual, replication and tumor formation in the cervical tissue may be reduced or prevented by administration of an IL-15 agonist or derivative thereof.

Moreover, administration of an IL-15 agonist or derivative thereof is also deemed advantageous in cases where a current treatment of a bacterial infection is supported. For example, where a drug resistant bacterial (preferably superficial) infection such as a MRSA infection is treated with bacteriophages that recognize and infect the pathogen in a bacterial antigen-specific manner, an IL-15 agonist or derivative thereof may support a NK and T cell immune response to the infection.

Additionally, it is also contemplated that administration of an IL-15 agonist or derivative thereof may provide various benefits to an individual prior to administration of an immune compromising therapy such as radiation therapy and/or chemotherapy. In such use, it is particularly preferred that an IL-15 agonist or derivative thereof is administered to the individual at a dose that stimulates formation of memory NK cells, T_{CM} central memory cells, T_{SCM} stem cell memory cells, and/or T_{EM} effector memory cells. Depending on the particular type, severity, and duration of the immune compromising therapy, leukapheresis may be performed to collect lymphocytes for transfusion to the individual after the immune compromising therapy. Most typically, leukapheresis is performed at least 12 hours, or at least 24 hours, or at least 36 hours, or at least 48 after administration of an IL-15 agonist or derivative thereof. Upon conclusion of the immune compromising therapy, the so isolated cells may be transfused back to the patient.

The IL-15 agonist or derivative thereof can be administered at a dosage to enhance immunity. Example dosages include about 0.1-1,000 μg/kg body weight, and more typically about 1.0-100 μg/kg body weight, or about 5-25 μg/kg body weight, or about 1-10 μg/kg body weight, or about 10-50 μg/kg body weight, or about 20-80 μg/kg body weight. Therefore, dosages for a single administration will typically be about 5-5,000 μg, or about 10-500 μg, or about 50-1,000 mcg, or about 200-1,500 μg, or about 500-2,500 μg, or about 1,000-3,000 mcg, or about 2,500-5,000 μg. Of course, dosages may also be adjusted within the above ranges according to one best tolerated by the individual.

Moreover, it should be noted that suitable dosages may also be based on a specific effect that is desired. For example, an individual may receive one or more dosages of an IL-15 agonist or derivative thereof to increase proliferative capacity of NK cells and/or T cells by at least 5% (relative to a point in time prior to administration of the stabilized IL-15 compounds), or at least 10%, or at least 15%, or at least 20%, or at least 30%, or at least 50%, or even higher. Likewise, the individual may receive one or more dosages of an IL-15 agonist or derivative thereof to increase cytotoxicity of NK cells and/or T cells by at least 5% (relative to a point in time prior to administration of an IL-15 agonist or derivative thereof), or at least 10%, or at least 15%, or at least 20%, or at least 30%, or at least 50%, or even higher. In another example, it is also contemplated that the individual may receive one or more dosages of an IL-15 agonist or derivative thereof to increase absolute numbers of various immune competent cells, including NK cells, T cells, various memory cells, and macrophages. Therefore, it should be appreciated that dosages for an IL-15 agonist or derivative thereof may be selected such that administration will change the n-tile rank for a specific immune competent cell population in the individual. For example, the dosage of an IL-15 agonist or derivative thereof may be selected such that the number of NK cells will increase from a bottom quintile or quartile rank to a top or second quintile or quartile rank. The skilled artisan will be readily apprised of such cell numbers and ranking based on known reference ranges for immune competent cells (see e.g., Data in Brief 12 (2017) 400-404).

Depending on the particular use and purpose, it is contemplated that administration of an IL-15 agonist or derivative thereof may be performed in a repetitive manner, with at least several days, or at least several weeks, or at least several months in between one administration and a subsequent administration. Therefore, administration of contemplated compositions can be done weekly, biweekly (every other week), monthly, bimonthly (every other month), quarterly, or at even longer intervals such as twice or once a year. As will be readily appreciated, suitable frequencies of administration will be readily determined by various manners well known in the art. For example, a blood count may provide cell counts for total lymphocytes and/or subsets thereof, such as NK cells, CD8⁺ T cells, CD4⁺ T cells, T_{EM} cells, etc. In another example, activity and/or proliferation capacity of NK cells, CD8⁺ T cells, CD4⁺ T cells, T_{EM} cells may be used to determine the frequency and/or dosage of the stabilized IL-15 compounds.

In addition, it should be appreciated that the methods contemplated herein may further be assisted with any suitable treatment modalities that are appropriate for treatment of a specific condition. Likewise, it should be recognized that additional therapeutics may be used in conjunction with the methods presented herein that systemically act on an individual to delay onset or slow down progression of age-related signs and symptoms. Therefore, contemplated additional agents include hormones, and especially those used in hormone replacement therapy for female individuals and testosterone (and precursors thereof) for male individuals, as well as human growth hormone for both male and female individuals.

In further embodiments, an IL-15 agonist, such as NAI or derivative thereof, and/or a pharmaceutical composition comprising an IL-15 agonist or derivative thereof, may be used to treat a viral infection, symptoms of the viral infection and/or secondary infections caused by the viral infection in a subject in need thereof. In particular embodiments, a viral infection is treated by contacting a cell, or a nucleus of a cell, with an effective amount of an IL-15 agonist or derivative thereof. In further embodiments, the cell is a T cell, NK cell, monocyte, macrophage, dendritic cell, keratinocyte, fibroblast, myocyte, and/or nerve cell. The IL-15 agonist or derivative thereof may be administered or otherwise provided in a composition, such as a pharmaceutical composition further comprising one or more pharmaceutically acceptable excipients as described herein. Additional pharmaceutical ingredients may also be administered concurrent or sequential with the IL-15 agonist or derivative thereof. In further embodiments, the viral infection can be inhibited in vitro or in vivo.

In further embodiments, the therapeutically effective amount of the IL-15 agonist or derivative thereof, can be an amount that treats a viral infection. In one embodiment, the therapeutically effective amount of the IL-15 agonist ranges between about 10 μg to about 500 μg per dose. In a particular embodiment, the therapeutically effective amount of the IL-15 agonist is about 50 μg per dose, about 100 μg per dose, about 200 μg dose, or about 400 μg per dose.

The therapeutically effective amount of the IL-15 or derivative thereof may be administered one or more times depending on the identity of the infecting virus and severity of the infection. For example, the IL-15 agonist or derivative thereof may be administered once per week, twice per week, three or more times per week, less than once per week, once every two weeks, once every three weeks, once every four or more weeks, semi-monthly, monthly, or bimonthly. U.S. 62/686,846 reports doses and regiments useful for using NAI to treat an HIV infection. The contents of U.S. 62/686,846 are herein incorporated by reference in its entirety.

The IL-15 agonist or derivative thereof may be administered to a subject suffering from or at risk of suffering from a viral infection. The viral infection or risk of viral infection can be caused by either DNA or RNA viruses, either of which may be single- or double-stranded. The virus may belong to one of the following families of viruses: herpesviridae, adenoviridae, polyomavididae, poxviridae, reoviridae, coronaviridae, picornaviridae, flaviviridae, hepeviridae, togaviridae, filoviridae, paramyxoviridae, pneumoviridae, rhabdoviridae, hantaviridae, and orthomyxoviridae. Particular viruses amenable to treatment by the IL-15 agonist or derivative thereof include, but are not limited to, human herpesvirus, varicella zoster virus, rotavirus, coronavirus (COVID-19; SARS-CoV-2), SARS virus, poliovirus, rhinovirus, hepatitis A virus, yellow fever virus, west nile virus, hepatitis C virus, dengue fever virus, zika virus, rubella virus, sindbis virus, Chikungunya virus, Ebola virus, Marburg virus, measles virus, mumps virus, respiratory syncytial virus, rabies virus, influenza virus A, influenza virus B, influenza virus C, and influenza virus D. In a specific embodiment, the virus is an influenza virus or a varicella zoster virus. In some instances, the subject is not infected by, or at risk of being infected by, HPV and/or HIV.

In a particular embodiment, an IL-15 agonist, such as NAI, or derivative thereof, or a pharmaceutical composition comprising the IL-15 agonist or derivative thereof may be used to treat a coronavirus infection, such as COVID-19 and/or SARS-CoV-2 infection, in a subject in need thereof. Additionally or alternatively, an IL-15 agonist, such as NAI or derivative thereof, or a pharmaceutical composition comprising the IL-15 agonist or derivative thereof may be used to treat symptoms of a coronavirus infection, such as coronavirus-induced lymphopenia. Additionally or alternatively, an IL-15 agonist, such as NAI or derivative thereof, or a pharmaceutical composition comprising the IL-15 agonist or derivative thereof may be used to treat a secondary infection resulting from a coronavirus infection, such as coronavirus-induced pneumonia.

Combination therapies are also provided herein. Thus, an IL-15 agonist, such as NAI, or a derivative thereof, and/or a pharmaceutical composition comprising an IL-15 agonist or derivative thereof, and one or more other active agents may be used to treat a viral infection, such as the viral infections disclosed herein, symptoms of a viral infection disclosed herein and/or a secondary infection caused by a virus infection disclosed herein in a subject in need thereof. Further active agents for use with an IL-15 agonist, such as NAI, or derivative thereof include one or more of an interleukin such as IL-1 and IL-6; a TNF-α antagonist/inhibitor such as infliximab, adalimumab, certolizumab pegol, golimumab and etanercept; a type 1 interferon (IFN) such as IFN-α and IFN-β; chloroquine and/or chloroquine phosphate; an anti-viral agent such as lopinavir, ritonavir and interferon alfa-2b; an antibiotic such as moxifloxacin; and/or a corticosteroid such as prednisone and prednisolone.

In a particular embodiment, a combination of NAI or derivative thereof and IL-1 is used/administered sequentially or concomitantly to treat a viral infection in a subject in need thereof.

In a further particular embodiment, a combination of NAI or derivative thereof and IL-6 is used/administered sequentially or concomitantly to treat a viral infection in a subject in need thereof.

In a further particular embodiment, a combination of NAI or derivative thereof and a TNF-α antagonist/inhibitor is used/administered sequentially or concomitantly to treat a viral infection in a subject in need thereof.

In a further particular embodiment, a combination of NAI or derivative thereof and a type 1 interferon is used/administered sequentially or concomitantly to treat a viral infection in a subject in need thereof.

In some embodiments, the combination therapies disclosed herein may be used to treat a viral infection caused by human herpesvirus, varicella zoster virus, rotavirus, coronavirus, SARS virus, poliovirus, rhinovirus, hepatitis A virus, yellow fever virus, west nile virus, hepatitis C virus, dengue fever virus, zika virus, rubella virus, sindbis virus, Chikungunya virus, Ebola virus, Marburg virus, measles virus, mumps virus, respiratory syncytial virus, rabies virus, influenza virus A, influenza virus B, influenza virus C, and influenza virus D. In a further particular embodiment, the viral infection may caused by coronavirus, influenza virus A, influenza virus B, influenza virus C, or influenza virus D.

In a particular embodiment, treatment of a coronavirus infection, such as COVID-19 and/or SARS-CoV-2 infection, and/or symptoms thereof, and/or a secondary infection caused by a coronavirus infection is provided herein and may comprise administering an IL-15 agonist, such as NAI, or derivative thereof, and/or a pharmaceutical composition comprising the IL-15 agonist or derivative thereof alone or in combination, either sequentially or concomitantly, with one or more an interleukin such as IL-1 and IL-6; a TNF-α antagonist/inhibitor such as infliximab, adalimumab, certolizumab pegol, golimumab and etanercept; a type 1 interferon (IFN) such as IFN-α and IFN-β; chloroquine and/or chloroquine phosphate; an anti-viral agent such as lopinavir, ritonavir and interferon alfa-2b; an antibiotic such as moxifloxacin; a corticosteroid such as prednisone and prednisolone, and/or oxygen treatment.

Separate dosage forms/active agents can optionally be co-packaged, for example in a single container or in a plurality of containers within a single outer package or co-presented in separate packaging (“common presentation”). As an example of co-packaging or common presentation, a kit is contemplated comprising, in separate containers, an IL-15 agonist, such as NAI or a derivative thereof, and one or more of the further active agents disclosed herein. In another example, the IL-15 agonist, such as NAI or a derivative thereof, and the one or more of the further active agents are separately packaged and available for sale independently of one another, but are co-marketed or co-promoted for use according to the invention. The separate dose forms/active agents may also be presented to a subject separately and independently, for use according to the invention.

### III. Compositions

In additional embodiments, pharmaceutical compositions are provided herein comprising an IL-15 agonist or derivative thereof, such as NAI, and a pharmaceutically-acceptable carrier. For example, an NAI may be formulated with a pharmaceutically-acceptable carrier. The compositions disclosed herein may be administered orally, parenterally, by inhalation spray, topically, rectally, nasally, buccally, vaginally, or via an implanted reservoir. The term “parenteral” as used herein includes subcutaneous, intravenous, intramuscular, intra-articular, intra-synovial, intrasternal, intrathecal, intrahepatic, intralesional, and intracranial injection or infusion techniques. In a particular embodiment, the compositions are administered orally, intraperitoneally or intravenously.

Of course, it should be appreciated that the composition/formulation may also be made such that an IL-15 agonist or derivative thereof will be released over time at lower continuous dosages (e.g., from a bioerodable or biodegradable polymer or other matrix). For example, an IL-15 agonist or derivative thereof may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, intravesicularly or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Most typically, treatment of human patients or other animals will be carried out using a therapeutically effective amount of an IL-15 agonist or derivative thereof in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with a desired preventive outcome. Generally, amounts will be in the range of those commonly used for N-803, although in certain instances lower amounts may be desired, for example due to repeated administration. Most typically, where subcutaneously or intradermally administered, an IL-15 agonist or derivative thereof may be injected at a single site or at multiple sites (especially where site specific reactions occur).

The compositions disclosed herein may be orally administered in any orally acceptable dosage form including, but not limited to, capsules, tablets, troches, elixirs, suspensions, syrups, wafers, chewing gums, aqueous suspensions, or solutions.

The oral compositions disclosed herein may contain additional ingredients such as: a binder such as microcrystalline cellulose, gum tragacanth or gelatin; an excipient such as starch or lactose, a disintegrating agent such as alginic acid, corn starch and the like; a lubricant such as magnesium stearate; a glidant such as colloidal silicon dioxide; and a sweetening agent such as sucrose or saccharin or flavoring agent such as peppermint, methyl salicylate, or orange flavoring. When the dosage unit form is a capsule, it may additionally contain a liquid carrier such as a fatty oil. Other dosage unit forms may contain other various materials which modify the physical form of the dosage unit, such as, for example, a coating. Thus, tablets or pills may be coated with sugar, shellac, or other enteric coating agents. A syrup may contain, in addition to the active ingredients, sucrose as a sweetening agent and certain preservatives, dyes and colorings and flavors. Materials used in preparing these various compositions should be pharmaceutically or veterinarally pure and non-toxic in the amounts used.

Additionally or alternatively, the compositions disclosed herein may be formulated for parenteral administration where the active ingredient may be incorporated into a solution or suspension. The solutions or suspensions may also include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

The pharmaceutical forms suitable for injectable use include sterile solutions, dispersions, emulsions, and sterile powders. The final form should be stable under conditions of manufacture and storage. Furthermore, the final pharmaceutical form should be protected against contamination and should, therefore, be able to inhibit the growth of microorganisms such as bacteria or fungi. A single intravenous or intraperitoneal dose can be administered. Alternatively, a slow long-term infusion or multiple short-term daily infusions may be utilized, typically lasting from 1 to 8 days. Alternate day dosing or dosing once every several days may also be utilized.

Sterile, injectable solutions may be prepared by incorporating a compound in the required amount into one or more appropriate solvents to which other ingredients, listed above or known to those skilled in the art, may be added as required. Sterile injectable solutions may be prepared by incorporating the compound in the required amount in the appropriate solvent with various other ingredients as required. Sterilizing procedures, such as filtration, may then follow. Typically, dispersions are made by incorporating the compound into a sterile vehicle which also contains the dispersion medium and the required other ingredients as indicated above. In the case of a sterile powder, the particular methods include vacuum drying or freeze drying to which any required ingredients are added.

Suitable pharmaceutical carriers include sterile water; saline, dextrose; dextrose in water or saline; condensation products of castor oil and ethylene oxide combining about 30 to about 35 moles of ethylene oxide per mole of castor oil; liquid acid; lower alkanols; oils such as corn oil; peanut oil, sesame oil and the like, with emulsifiers such as mono- or di-glyceride of a fatty acid, or a phosphatide, e.g., lecithin, and the like; glycols; polyalkylene glycols; aqueous media in the presence of a suspending agent, for example, sodium carboxymethylcellulose; sodium alginate; poly(vinylpyrolidone); and the like, alone, or with suitable dispensing agents such as lecithin; polyoxyethylene stearate; and the like. The carrier may also contain adjuvants such as preserving stabilizing, wetting, emulsifying agents and the like together with the penetration enhancer. In all cases, the final form, as noted, must be sterile and should also be able to pass readily through an injection device such as a hollow needle. The proper viscosity may be achieved and maintained by the proper choice of solvents or excipients. Moreover, the use of molecular or particulate coatings such as lecithin, the proper selection of particle size in dispersions, or the use of materials with surfactant properties may be utilized.

U.S. Pat. Nos. 5,916,596, 6,506,405, and 6,537,579 teach the preparation of nanoparticles from the biocompatible polymers, such as albumin. Thus, provided herein are methods for the formation of nanoparticles by a solvent evaporation technique from an oil-in-water emulsion prepared under conditions of high shear forces (e.g., sonication, high pressure homogenization, or the like).

Additionally or alternatively, the compositions disclosed herein may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Additionally or alternatively, the compositions disclosed herein may be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Additionally or alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Additionally or alternatively, the compositions disclosed herein may be formulated for ophthalmic use as micronized suspensions in isotonic, pH adjusted sterile saline, or as solutions in isotonic, pH adjusted sterile saline, either with or without a preservative such as benzylalkonium chloride. Additionally or alternatively, for ophthalmic uses, the compositions may be formulated in an ointment such as petrolatum.

Additionally or alternatively, the compositions disclosed herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

### FURTHER EMBODIMENTS

Embodiment 1: A method of treating age-related decline of activity of immune competent cells in a healthy individual is provided, comprising: administering to the individual a composition that includes an IL-15 agonist or derivative thereof a dose that increases proliferative capacity and/or cytotoxicity of a NK cell or a T cell.

Embodiment 2: A method of preventing or reducing likelihood of an infectious disease or cancer in a healthy individual, comprising: administering to the individual a composition that includes a stabilized IL-15 compound at a dose that increases proliferative capacity and/or cytotoxicity of a NK cell or a T cell.

Embodiment 3: A method of enhancing longevity in an individual, comprising: administering to the individual a composition that includes a IL-15 agonist or derivative thereof at a dose that increases proliferative capacity and/or cytotoxicity of a NK cell or a T cell.

Embodiment 4: A method of enhancing vaccine protection of a healthy individual, comprising: administering to the individual a vaccine composition that comprises an antigen of a pathogenic virus or pathogenic bacterium; and administering a IL-15 agonist or derivative thereof to the healthy individual within equal or less than 14 days from the step of administering the vaccine composition.

Embodiment 5: A method of suppressing viral activation in an individual infected with a latent virus, comprising: administering to the individual a composition that includes a IL-15 agonist or derivative thereof at a dose that prevents viral activation in the individual.

Embodiment 6: A method of treating an individual prior to administration of an immune compromising therapy, comprising: administering to the individual a composition that includes a IL-15 agonist or derivative thereof at a dose that stimulates formation of memory NK cells, T_{CM} central memory cells, T_{SCM} stem cell memory cells, and/or T_{EM} effector memory cells.

Embodiment 7: The method of any one of the previous embodiments, wherein the individual is at least 50 years of age, at least 55 years of age, or is at least 60 years of age.

Embodiment 8: The method of any one of the previous embodiments, wherein the individual has a total T cell count that is at or below a bottom quartile for a reference range for the total T cell count.

Embodiment 9: The method of any one of the previous embodiments, wherein the individual has a total NK cell count that is at or below a bottom quartile for a reference range for the total NK cell count.

Embodiment 10: The method of any one of the previous embodiments, wherein the individual has a total memory cell count that is at or below a bottom quartile for a reference range for the total memory cell count.

Embodiment 11: The method of any one of the previous embodiments, wherein the IL-15 agonist or derivative thereof comprises a IL-15N72D:IL-15RαSu/IgG1 Fc complex.

Embodiment 12: The method of any one of the previous embodiments, wherein the IL-15 agonist or derivative thereof comprises N-803.

Embodiment 13: The method of any one of the previous embodiments wherein the step of administering comprises subcutaneous injection of the IL-15 agonist or derivative thereof.

Embodiment 14: The method of any one of the previous embodiments, wherein the step of administering comprises multiple subcutaneous injections of the IL-15 agonist or derivative thereof at a monthly or quarterly schedule.

Embodiment 15: The method of any one of the previous embodiments, wherein the dose of the IL-15 agonist or derivative thereof is 1-10 mcg/kg or 10-25 mcg/kg.

Embodiment 16: The method of any one of the previous embodiments, wherein the individual is a healthcare professional, a teacher, or at least 50 years of age.

Embodiment 17: The method of any one of the previous embodiments, wherein the individual is predisposed to infectious diseases or cancer and/or has a family history of predisposition to infectious diseases or cancer.

Embodiment 18: The method of any one of the previous embodiments, wherein the IL-15 agonist or derivative thereof is administered to increase a total T cell count by at least 10%.

Embodiment 19: The method of any one of the previous embodiments, wherein the IL-15 agonist or derivative thereof is administered to increase a total NK cell count by at least 10%.

Embodiment 20: The method of embodiment 4, wherein the pathogenic virus is an influenza virus, a morbillivirus, a rubeola virus, a rubella virus, a varicella virus, a hemorrhagic fever virus, an enterovirus, and a hepatitis virus.

Embodiment 21: The method of embodiment 4, wherein the pathogenic bacterium is Mycobacterium tuberculosis, Corynebacterium diphtheriae, Clostridium tetani, Bordetella pertussis, Haemophilus influenzae type B, Vibrio cholerae, Salmonella Typhi, or Streptococcus pneumoniae.

Embodiment 22: The method of embodiment 5, wherein the latent virus is a dormant retrovirus that is integrated into a host cell genome of the individual.

Embodiment 23: The method of embodiment 5, wherein the latent virus is a dormant varicella zoster virus that is integrated into a host cell of the individual.

Embodiment 24: The method of embodiment 5, wherein the latent virus is a human papillomavirus that is integrated into a host cell of the individual.

Embodiment 25: The method of embodiment 6, wherein the immune compromising therapy is radiation therapy or chemotherapy.

Embodiment 26: The method of embodiment 6, further comprising a step of subjecting the individual to a leukapheresis to collect lymphocytes for transfusion to the individual after the immune compromising therapy.

### EXAMPLES

The following examples are provided to further illustrate the invention disclosed herein but, of course, should not be construed as in any way limiting its scope.

### Example 1: NAI

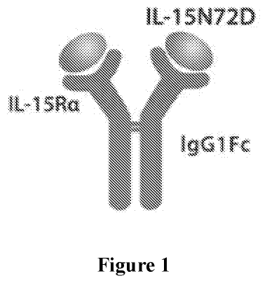

NAI is an IL-15 superagonist complex comprising the IL-15N72D derivative bound to an IL-15Rα/IgG1 Fc fusion protein (previously described in U.S. Pat. No. 9,328,159) and can be seen in FIG. 1.

### Example 2: Treatment of Viral Infections

Subjects were treated with Bacillus Calmette Guerin (BCG) vaccine alone or co-administered with NAI. The incidence of various infections was determined following BCG monotherapy and co-administration of BCG and NAI. As shown in Table 1, addition of an IL-15 agonist to the BCG vaccine reduced the incidence of viral infections.

TABLE 1

| Condition | BCG alone (N = 59) | BCG + NAI (N-57) |
|---|---|---|
| Infections and Infestations | 18 (31%) | 14 (25%) |
| Acute sinusitis | 1 (2%) | 0 |
| Bronchitis | 1 (2%) | 0 |
| Conjunctivitis | 1 (2%) | 0 |
| Cystitis | 1 (2%) | 2 (4%) |
| Disseminated BCG infection | 1 (2%) | 0 |
| Diverticulitis | 1 (2%) | 0 |
| Eye infection | 1 (2%) | 0 |
| Gastroenteritis viral | 1 (2%) | 0 |
| Herpes zoster | 1 (2%) | 0 |
| Hordeolum | 1 (2%) | 0 |
| Influenza | 1 (2%) | 0 |
| Kidney infection | 1 (2%) | 0 |
| Lower respiratory tract | 0 | 1 (2%) |
| infection | | |
| Nasopharyngitis | 2 (3%) | 0 |
| Prostate infection | 0 | 1 (2%) |
| Pseudomonas infection | 0 | 1 (2%) |
| Pyelonephritis | 0 | 1 (2%) |
| Sinusitis | 0 | 1 (2%) |
| Upper respiratory tract | 1 (2%) | 3 (5%) |
| infection | | |
| Urinary tract infection | 9 (15%) | 8 |
| | | (14%) |
| Viral sinusitis | 1 (2%) | 0 |
| Wound infection | 1 (2%) | 0 |

### Example 3

Among other suitable stabilized IL-15 compounds, N-803 (human IL-15N72D:IL-15RαSu/IgG1 Fc complexes) is especially preferred. Most typically, administration will be by subcutaneous injection using a monthly, quarterly, or once yearly schedule at a dosage of between 1-25 mcg/kg patient weight. N-803 can be obtained from Altor Bioscience (2810 North Commerce Parkway, Miramar, Fla. 33025-3958).

Cell analysis before and after administration can be done using heparinized whole blood samples collected at a clinical site. Aliquots of fresh whole blood can be used for real time antibody labeling for flow cytometric analyses and the remainder of the samples is processed to plasma and PBMC using standard Ficoll-Hypaque isolation immediately upon receipt. PBMC is cryopreserved in 10% DMSO (Sigma, St. Louis, Mo.) and 12.5% HAS (Gemini, Atlanta, Ga.) at −80° C. and subsequently maintained in vapor phase liquid nitrogen freezers. Serum is collected at the site within 4 hours of blood draw and frozen at −80° C.

Whole blood flow cytometric analyses for immunophenotyping will be performed using fresh blood samples. Fresh whole blood samples are labeled with fluorescently-labeled antibodies to cell surface molecules CD45 (21D1), CD3 (UCHT1), CD8 (SKi), CD56 (NCAM16.1), CD16 (3G8), CD14 (MOP9), CD123 (9FS) (all BD Biosciences, San Jose, Calif.) and CD4 (RPA-T4), CD19 (HIB19), and HLA-DR (L243) (all Biolegend, San Diego, Calif.) using a method adapted from Hensley et al. Samples are treated with BD FACS™ Lysing Solution (BD Biosciences) and will be immediately frozen at −80° C. for later batch testing on a BD LSRII flow cytometer. Absolute cell numbers can be obtained using Trucount tubes (BD Biosciences). Presence of intracellular Ki-67 can be analyzed using thawed PBMC labeled with antibodies to CD14 (MoP9, exclusion marker), CD56 (NCAM16.2), CD4 (SK3), CD8 (SKi) (all, BD Biosciences), CD3 (SK7), Ki67 (both, Biolegend) as well as Fixable Viability Dye eFluor 780 and FoxP3/Transcription Factor Staining Buffer Set (both eBioscience, San Diego, Calif.).

Serum N-803 concentrations can be assessed using a human IL-15 specific ELISA kit (R & D Systems, Minneapolis, Minn.) and ALT-803 for generation of the standard curve. Mean values of triplicate wells will be reported.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Particular embodiments of this invention are described herein, including the best mode known to the inventors for carrying out the invention. Variations of those particular embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method for preventing, reducing the occurrence of, or treating a viral infection in a subject in need thereof, the method comprising administering to the subject a therapeutically effective amount of an IL-15 agonist, wherein the viral infection is not an HIV infection or an HPV infection.

2. The method of claim 1, wherein the IL-15 agonist is nogapendekin alfa-imbakicept (NAI).

3. The method of any one of the previous claims, wherein the IL-15 agonist is administered at least once per week.

4. The method of claim 3, wherein the IL-15 agonist is administered once per week.

5. The method of claim 3, wherein the IL-15 agonist is administered twice per fortnight.

6. The method of claim 3, wherein the IL-15 agonist is administered once every three weeks.

7. The method of any one of the previous claims, wherein the therapeutically effective amount of the IL-15 agonist is between about 10 μg and about 500 μg per dose.

8. The method of claim 7, wherein the therapeutically effective amount of the IL-15 agonist is about 50 μg per dose.

9. The method of any one of the previous claims, wherein the IL-15 agonist is administered subcutaneously or intravenously.

10. The method of any one of the previous claims, wherein the subject is human.

11. The method of any one of the previous claims, wherein the viral infection is caused by a DNA virus.

12. The method of claim 11, wherein the DNA virus is selected from the group consisting of papillomaviridae, herpesviridae, adenoviridae, polyomavididae, and poxviridae.

13. The method of claim 12, wherein the DNA virus is selected from the group consisting of human papilloma virus, human herpesvirus, and varicella zoster virus.

14. The method of any one of claims 1-10, wherein the viral infection is caused by an RNA virus.

15. The method of claim 14, wherein the RNA virus is selected from the group consisting of reoviridae, coronaviridae, picornaviridae, flaviviridae, hepeviridae, togaviridae, filoviridae, paramyxoviridae, pneumoviridae, retroviridae, rhabdoviridae, hantaviridae, and orthomyxoviridae.

16. The method of claim 15, wherein the RNA virus is selected from the group consisting of rotavirus, coronavirus, SARS virus, poliovirus, rhinovirus, hepatitis A virus, yellow fever virus, west nile virus, hepatitis C virus, dengue fever virus, zika virus, rubella virus, sindbis virus, Chikungunya virus, Ebola virus, Marburg virus, measles virus, mumps virus, respiratory syncytial virus, rabies virus, human immunodeficiency virus, influenza virus A, influenza virus B, influenza virus C, and influenza virus D.

17. The method of claim 16, wherein the RNA virus is coronavirus, human immunodeficiency virus, influenza virus A, influenza virus B, influenza virus C, or influenza virus D.

18. The method of any one of the previous claims further comprising administering to the subject a therapeutically effective amount of one or more of an interleukin; a TNF-α antagonist; a type 1 interferon (IFN); chloroquine and/or chloroquine phosphate; an anti-viral agent; an antibiotic; and/or a corticosteroid such as prednisone and prednisolone.

19. The method of claim 18, wherein the interleukin is IL-1 and/or IL-6.

20. The method of claim 18, wherein the TNF-α antagonist is infliximab, adalimumab, certolizumab pegol, golimumab and/or etanercept.

21. The method of claim 18, wherein the type 1 interferon (IFN) is IFN-α and/or IFN-β.

22. The method of claim 17, wherein the coronavirus is coronavirus disease 2019 (COVID-19) virus and/or or severe acute respiratory syndrome coronavirus 2 (SARS-CoV-2).

23. The method of claim 22, further comprising administering to the subject a therapeutically effective amount of one or more of chloroquine; chloroquine phosphate; an anti-viral agent; an antibiotic; a corticosteroid, and/or oxygen to the subject in need thereof.

24. The method of any one of the previous claims, wherein activation of the virus is suppressed.

25. A method for enhancing an immune response or preventing or reducing the likelihood of cancer in a healthy individual, the method comprising administering to the individual a composition comprising an IL-15 agonist at a dose that increases the proliferative capacity and/or cytotoxicity of a NK cell or a T cell.

26. The method of claim 25, wherein the IL-15 agonist is NAI.

27. The method of any one of claims 25-26, wherein the individual is at least 50 years of age, at least 55 years of age, or at least 60 years of age.

28. The method of any one of claims 25-27, wherein the individual has one or more of the following:
a. a total T cell count that is at or below a bottom quartile for a reference range for the total T cell count;
b. a total NK cell count that is at or below a bottom quartile for a reference range for the total NK cell count; and/or
c. a total memory cell count that is at or below a bottom quartile for a reference range for the total memory cell count.

29. The method of any one of claims 25-28, wherein the individual is a healthcare professional or a teacher.

30. The method of any one of claims 25-29, wherein the individual is predisposed to infectious diseases or cancer and/or has a family history of predisposition to infectious diseases or cancer.

31. The method of any one of claims 25-30, wherein the IL-15 agonist is administered within 14 days of administration of a vaccine composition.

32. The method of any one of claims 25-31, wherein viral activation of a latent virus is suppressed.

33. The method of claim 32, wherein the latent virus is a dormant varicella zoster virus or a human papillomavirus.

34. The method of any one of claims 25-33, wherein the IL-15 agonist is administered prior to administration of an immune compromising therapy.

35. The method of claim 34, wherein the immune compromising therapy is radiation therapy or chemotherapy.

36. The method of any one of claims 25-35, wherein the IL-15 agonist is administered to increase a total T cell count and/or a total NK cell count by at least 10%.

37. The method of any one of claims 25-36, wherein the step of administering comprises subcutaneous injection of the IL-15 agonist.

38. The method of claim 37, wherein the step of administering comprises multiple subcutaneous injections of the IL-15 agonist at a monthly or quarterly schedule.

39. The method of any one of claims 25-38, wherein the therapeutically effective amount of the IL-15 agonist is about 1-10 μg/kg per dose.

40. The method of any one of claims 25-39, wherein the therapeutically effective amount of the IL-15 agonist is about 10-25 μg/kg per dose.
